(19) United States
(12) Patent Application Publication
(10) Pub. No.: US 2026/0142037 A1
(43) Pub. Date: May 21, 2026

(54) GENISCi Whole-Health Intelligence System and Methods

(71) Applicant: Rezaul Abid, Aldie, VA (US)

(72) Inventor: Rezaul Abid, Aldie, VA (US)

(73) Assignee: GENISCi LLC, Leesburg, VA (US)

(21) Appl. No.: 19/386,680

(22) Filed: Nov. 12, 2025

Related U.S. Application Data
(60) Provisional application No. 63/722,852, filed on Nov. 20, 2024.

(51) Int. Cl. G16H 50/30; G06F 18/25; G10L 25/63; G16H 10/60

(52) U.S. Cl. CPC G16H 50/30; G06F 18/251; G10L 25/63; G16H 10/60

(57) ABSTRACT

A computer-implemented whole-health intelligence system and method providing personalized clinical and integrative health guidance based on multimodal data including medical history, lifestyle behaviors, psychosocial factors, environmental inputs, and biological metrics. The system constructs a hybrid digital health twin combining graph-based physiological relationships and vectorized embeddings, then generates personalized recommendations via generative AI models and clinical rule-based logic. Safety gating logic evaluates risk patterns and escalates medically significant findings to clinician review. The system supports longitudinal health optimization, annual precision-health evaluations, home-care coordination, and clinician co-pilot review workflows.

### CROSS-REFERENCE TO RELATED APPLICATIONS

This application claims priority to U.S. Provisional Ser. No. 63/722,852, filed Nov. 20, 2024, titled “Generative AI-Based Holistic Health Sciences Platform for Personalized Whole Health Management”. The entire contents of the above application are incorporated herein by reference.

### FIELD OF THE INVENTION

This invention relates to artificial intelligence systems in healthcare, and more specifically to integrated medical and behavioral guidance systems using hybrid patient modeling, clinical safety gating, and longitudinal precision-health optimization.

### BACKGROUND

The current healthcare paradigm is fragmented, reactive, episodic, and lacks integrated support for preventive, lifestyle, behavioral, and longitudinal health management. Existing systems fail to integrate clinical+lifestyle+psychosocial+behavioral data, provide whole-person guidance with safety gates, deliver continuous, adaptive health plans, support both clinical and home-care pipelines, enable collaborative patient-provider planning and review, and maintain safe, auditable reinforcement learning. Thus, a gap exists for a regulatory-compliant, AI-enabled whole-health system that continuously learns and assists patients, clinicians, and caregivers across clinical and integrative care contexts.

### SUMMARY OF THE INVENTION

The invention provides: a multimodal health data intake engine, hybrid digital health twin (graph+vector+temporal), clinical rule-engine+generative AI fusion, safety gating+clinician escalation, daily+annual precision guidance, controlled reinforcement loop, and caregiver & home-care data pathway.

### BRIEF DESCRIPTION OF DRAWINGS

FIG. 1—System architecture; FIG. 2—Safety-gated recommendation flow; FIG. 3—Annual precision-health review cycle; FIG. 4—Multi-role health support ecosystem; FIG. 5—Home-care data ingestion embodiment; FIG. 6—Personalized daily recommendation example; FIG. 7—Annual precision-health summary example; FIG. 8—Clinician co-pilot validation interface.

### DETAILED DESCRIPTION

Data intake layer receives clinical, patient-reported, psychosocial, and biometric data. Hybrid digital health twin combines graph relationships, vector embeddings, temporal memory, and personal health objectives. Inference engine combines generative AI and clinical rules.

### Safety System Screens High-Risk Patterns and Triggers Clinician Escalation

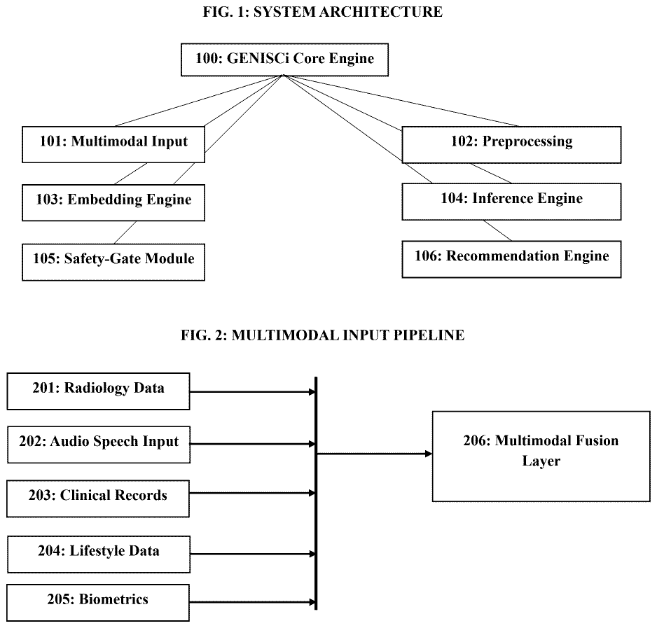

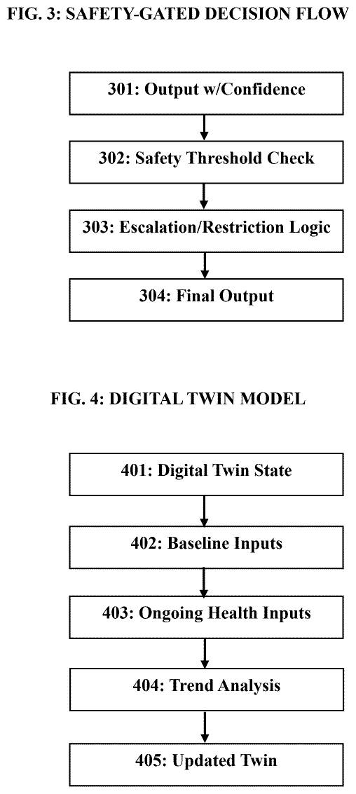

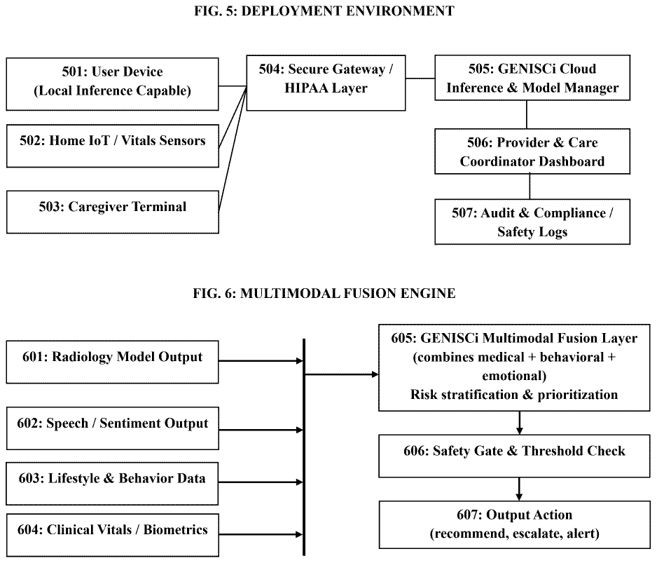

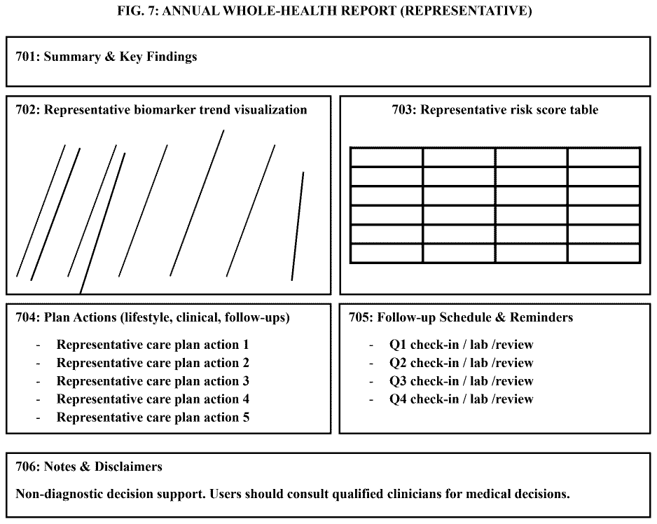

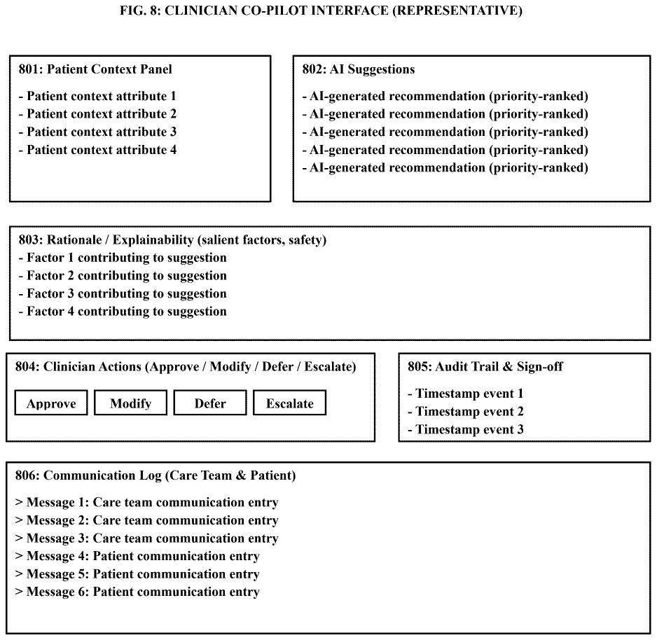

Recommendation engine produces daily micro plans and annual reports. Learning module updates safely under human control. (see FIG. 1) (see FIG. 2) (see FIG. 3) (see FIG. 4) (see FIG. 6)

### Safety Gating Across Imaging and Voice Modalities

Upon detection of clinically relevant anomalies in imaging or emotional distress signals in audio, the system triggers safety gating modalities (see FIG. 7; FIG. 8) including escalation pathways, clinician review alerts, in-home caregiver notification, or mental health support prompts.

### Audio Emotion and Sentiment Analysis Embodiment

In certain embodiments, the system receives voice input from a user, performs speech-to-text transcription, executes sentiment polarity classification, computes multi-class emotional states (happiness, sadness, neutrality, anger), and synthesizes a hybrid emotional inference score. Example: transcript indicates distress; sentiment negative; emotional state neutral (0.35 hybrid score).

### Multimodal Medical Imaging Embodiment

In certain embodiments, the system receives radiologic inputs including chest radiography, abdominal computed tomography, ultrasound, or future imaging modalities. The system preprocesses imaging data, performs anomaly detection using convolutional neural networks or transformer-based models, computes class probabilities, and generates labeled inferences with confidence scores (see FIG. 7; FIG. 8). Example outputs include Normal lung (1.00 confidence), Viral pneumonia (0.99 confidence), COVID-19 pneumonia (1.00 confidence), kidney stone (0.62 confidence), and medullary sponge kidney (0.97 confidence).

### Home-Care Use

System supports in-home caregivers contributing structured observations with clinician review when needed. (see FIG. 4; FIG. 5)

### Continuation Statement

Future embodiments may incorporate genomic, metabolomic, microbiome, sensor fusion, and imaging inputs. (see FIG. 3) The scope of the present disclosure encompasses these and other equivalent embodiments.

### End of Detailed Description

Numerous embodiments may be made. The scope of the invention is defined by the claims.

1. A computer-implemented whole-health intelligence system, herein referred to as the GENISCi platform, comprising one or more processors and memory storing instructions that, when executed, cause the system to:
(a) receive multimodal health inputs comprising:
(i) radiologic imaging data,
(ii) audio and speech data,
(iii) lifestyle and behavioral information, and
(iv) physiologic or biomarker data;
(b) generate an imaging inference including anomaly classification and an associated confidence score;
(c) generate sentiment and affect classification from the audio and speech data and compute a hybrid distress score;
(d) perform multimodal fusion of the imaging inference, hybrid distress score, lifestyle and behavioral inputs, and physiologic inputs to compute a unified whole-health risk score;
(e) apply a safety-gated clinical decision module configured to evaluate the unified risk score and determine whether to:
(i) deliver personalized lifestyle or wellness guidance to the user;
(ii) transmit an alert to trained support personnel or in-home care assistants; or
(iii) escalate for review by a licensed healthcare professional;
(f) update a longitudinal digital-twin profile based on historical and incoming multimodal health data; and
(g) generate an audit log comprising the multimodal inputs, inferences, safety gate decisions, and resulting actions;
wherein the platform is aligned with clinical practice environments, regulatory safeguards, and patient-centric workflows while leveraging multimodal neural architectures, longitudinal digital-twin modeling, and explainable artificial intelligence pipelines.

2. A computer-implemented method for delivering whole-health intelligence, comprising:
(a) receiving multimodal health inputs comprising radiologic imaging data, audio speech data, lifestyle information, and physiologic biomarker data;
(b) generating an imaging inference with anomaly classification and confidence score;
(c) generating sentiment and affect classification and a hybrid distress score from the audio speech data;
(d) performing multimodal fusion to compute a unified whole-health risk score;
(e) applying a safety-gated rules engine to determine whether to:
(i) provide a personalized wellness recommendation,
(ii) notify a trained in-home care assistant, or (iii) escalate for review by a licensed healthcare professional;
(f) updating a longitudinal digital-twin profile; and
(g) recording an audit trail comprising multimodal inputs, inferences, gating decisions, and actions.

3. A non-transitory computer-readable medium storing instructions that, when executed by one or more processors, cause performance of the method of claim 2.

4. The system of claim 1, wherein multimodal fusion is performed via a bus-style data layer aggregating modality-specific embeddings.

5. The system of claim 1, wherein the imaging module classifies lung normality, viral pneumonia, COVID-19 pneumonia, kidney stones, or medullary sponge kidney.

6. The system of claim 1, wherein affect inference comprises sentiment polarity and multi-class emotional state scores combined into a hybrid distress index.

7. The method of claim 2, wherein escalation occurs when the confidence score falls below a threshold.

8. The system of claim 1, further comprising a clinician co-pilot interface configured to enable licensed healthcare professionals to approve, modify, or reject suggested actions.

9. The system of claim 1, wherein explainability outputs identify salient image and audio features contributing to the risk score.

10. The method of claim 2, wherein the audit log supports HIPAA-compliant traceability.

11. The system of claim 1, wherein at least one inference executes on-device or at an edge appliance.

12. The system of claim 1, wherein user data is encrypted and de-identified prior to cloud transmission.

13. The method of claim 2, further comprising scheduling a telehealth review.

14. The system of claim 1, wherein caregiver notifications include task guidance for in-home support.

15. The method of claim 2, further comprising generating a caregiver task checklist.

16. The system of claim 1, wherein the digital twin generates an annual precision-health report.

17. The method of claim 2, wherein the report includes biomarker trends, lifestyle adherence, and expected physiologic trajectory.

18. The system of claim 1, wherein the digital-twin model adapts based on user feedback.

19. The system of claim 1, wherein the platform produces educational content or lifestyle guidance messages.

20. The medium of claim 3, wherein the instructions cause rendering of a whole-health dashboard for the user.
